SEQUENCE LISTING (4 sequences)

SEQ ID NO: 1 - DNA, 20 bp, Artificial Sequence
  Description of Artificial Sequence Synthetic modified oligonucleotide
  modified_base (11)..(11): 1-beta-D-arabinofuranosylcytosine
  See specification as filed for detailed description of substitutions and preferred embodiments
ggtggtggttctggtggtgg

SEQ ID NO: 2 - DNA, 20 bp, Artificial Sequence
  Description of Artificial Sequence Synthetic modified oligonucleotide
  modified_base (10)..(11): 1-beta-D-arabinofuranosylcytosine
  See specification as filed for detailed description of substitutions and preferred embodiments
ggtggtggtcctggtggtgg

SEQ ID NO: 3 - DNA, 21 bp, Artificial Sequence
  Description of Artificial Sequence Synthetic modified oligonucleotide
  modified_base (1)..(1): 1-beta-D-arabinofuranosylcytosine
  See specification as filed for detailed description of substitutions and preferred embodiments
cggtggtggttgtggtggtgg

SEQ ID NO: 4 - DNA, 22 bp, Artificial Sequence
  Description of Artificial Sequence Synthetic modified oligonucleotide
  modified_base (1)..(2): 1-beta-D-arabinofuranosylcytosine
  See specification as filed for detailed description of substitutions and preferred embodiments
ccggtggtggttgtggtggtgg

(19) United States
(12) Patent Application Publication
(10) Pub. No.: US 2023/0192752 A1
(43) Pub. Date: Jun. 22, 2023

(54) THERAPEUTIC AGENT FOR BLOOD CANCER

(71) Applicants: Aptabio Therapeutics Inc., Gyeonggi-do (KR); Samjin Pharmaceutical Co., Ltd., Seoul (KR)

(72) Inventors: Sung Hwan Moon, Gyeonggi-do (KR); Soo Jin Lee, Gyeonggi-do (KR); Yu Mi Ji, Gyeonggi-do (KR); Hee Jong Shin, Gyeonggi-do (KR); Min Hyo Ki, Gyeonggi-do (KR); Yong Bin Park, Gyeonggi-do (KR); Ji Hyun Um, Gyeonggi-do (KR)

(73) Assignees: Aptabio Therapeutics Inc., Gyeonggi-do (KR); Samjin Pharmaceutical Co., Ltd., Seoul (KR)

(21) Appl. No.: 16/609,367

(22) PCT Filed: Nov. 30, 2018
(86) PCT No.: PCT/KR2018/015054

§ 371 Date: Oct. 29, 2019

(30) Foreign Application Priority Data
Dec. 1, 2017 (KR) 10-2017-0164409
Nov. 29, 2018 (KR) 10-2018-0150255

(51) Int. Cl. C07H 21/02; A61K 9/00; A61K 9/08; A61K 47/22; A61P 35/02

(52) U.S. Cl. CPC C07H 21/02; A61K 9/0019; A61K 9/08; A61K 47/22; A61P 35/02

(57) ABSTRACT

Disclosed is an oligonucleotide-modified nucleic acid containing at least one 1-β-D-arabinofuranosylcytosine as a modified nucleic acid having therapeutic efficacies and guanosine. More particularly, a novel oligonucleotide-modified nucleic acid containing at least one modified nucleic acid (N) having therapeutic efficacies and being rich in guanosine (G) is synthesized and the fact that the novel oligonucleotide-modified nucleic acid has excellent apoptotic activities on blood cancer cells and drug-resistant blood cancer cells is identified. Based on this, provided is a composition for preventing, ameliorating or treating blood cancer, containing the novel oligonucleotide-modified nucleic acid, and the novel oligonucleotide-modified nucleic acid or a pharmaceutically acceptable salt thereof as an active ingredient.

### TECHNICAL FIELD

The present invention relates to a composition for preventing, ameliorating or treating blood cancer containing a novel oligonucleotide-modified nucleic acid having at least one 1-β-D-arabinofuranosylcytosine as a modified nucleic acid having therapeutic efficacies or a pharmaceutically acceptable salt thereof as an active ingredient.

### BACKGROUND ART

Blood (hematologic) cancer refers to cancer in which various blood cells are transformed into cancer cells, and the type of cancer that attacks the blood, bone marrow, and/or lymphatic system. This type of cancer includes leukemia, lymphoma, and multiple myeloma. Leukemia is blood cancer in which cancer cells transformed from hematopoietic stem cells producing blood cells cause overproduction of leukemia cells, but prevent proper production of normal blood cells, leading to infection, anemia, bleeding and the like. Lymphoma is a cancer that occurs in the lymphatic system, which includes non-Hodgkin's lymphoma and Hodgkin's lymphoma. Multiple myeloma is a blood cancer caused by abnormal differentiation and proliferation of plasma cells, which is a kind of white blood cells in the blood. More specifically, blood cancer is selected from the group consisting of non-Hodgkin's lymphoma, Hodgkin's lymphoma, multiple myeloma, leukemia, lymphoma, myelodysplastic syndrome, acute lymphoblastic leukemia, acute myelogenous leukemia, chronic lymphocytic leukemia, chronic myelogenous leukemia, solitary myeloma and aplastic anemia.

Each year, new cases of leukemia, Hodgkin's and non-Hodgkin's lymphoma and myeloma account for almost 10% of all new cancer cases diagnosed. In an aging society, the number of elderly people suffering from blood cancer is increasing, but their survival rate is very low. In particular, the survival rate of patients 65 years or older is only 9.4% and the survival rate of patients 80 years or older is as significantly low as 0% (Blood, 2012, 120;1165-1174).

Blood (hematologic) cancer is a disease caused by transformation into cancer cells from mutation cells created in the blood and lymphatic system while the mutation cells move all over the body, and there are about 70 types of various blood cancer. Therefore, patients diagnosed with blood cancer first receive remission induction chemotherapy to remove all of the immune cells from the body in order to kill mutant abnormal cells. Thereafter, consolidation chemotherapy including injection of toxic chemicals only into the abnormal cells is conducted. Remission induction chemotherapy is carried out by high-dose administration of a combination of highly cytotoxic anticancer drugs, which are very toxic and adverse and thus are inapplicable to administration to older patients. This is the reason why the viability of elderly patients with blood cancer is very low (Blood, 2010,116:5818˜5823).

In addition, there is no therapeutic effect on blood cancer because blood cancer is not responsive even to high-dose remission induction chemotherapy, or there are highly unmet medical needs associated with blood cancer because of a high resistance rate and a high recurrence rate within five years.

Currently, target therapies using antibody and kinase inhibitors have been developed, but the treatment of blood cancer still heavily depends upon chemotherapy and radiotherapy (The New England Journal of Medicine, 2014, 371:1005˜1015). Anticancer drugs used as chemotherapeutic agents for blood cancer are cytotoxic anticancer drugs with considerable toxicity, which are administered at very high doses due to low bioavailability and difficulty of target delivery to cancer cells. This causes serious side effects to patients, thus making treatment difficult. Therefore, there is a need for novel therapeutic agents having less toxicity and effective therapeutic efficacies.

Guanosine-rich oligonucleotides are known to have inhibitory effects against cell growth in a wide range of cancer cells, and function to regulate the cell cycle by binding to specific proteins in cells, for example, proteins important for cell growth and death specific oligonucleotides such as eEF1A, JNK, Ki-ras, nucleolin, stat3, telomerase and topoisomerase proteins, when treating cancer cells with the same, and these proteins are known to be over-expressed in cancer cells more than in normal cells (Christopher R. Ireson et al. Molecular cancer therapy, 2006, 2957-2962; Naijie Jing et al. Cancer research, 2004, 6603-6609; Christophe Marchand et al. The Journal of Biological Chemistry, 2002, 8906-8911).

These guanosine-rich oligonucleotides have special structural features in addition to triple hydrogen bonding to cytosine. Guanosine-rich oligonucleotides can have a four-stranded structure through intramolecular bonding or intermolecular bonding. Instead of forming a double helix structure through a general hydrogen bond between adenosine and thiamine, and between guanosine and cytidine, four guanosines are positioned on one plane to form Hoogsteen-type hydrogen bonds, which constitute a G-quadruplex. Two or more of such G-quadruplexes are continuously positioned to form a tetrahelical structure. In general, there are difficulties in developing oligonucleotides into drugs due to their low stability in blood and their low cell permeability. However, oligonucleotides constituting such a G-quadruplex are known to have relatively high blood stability and cell permeability due to structural characteristics thereof.

As described in U.S. Pat. No. 7,314,926, and U.S. Patent Publication No. 2007-105805, the oligonucleotides constituting such a G-quadruplex are known to bind to specific proteins that are highly expressed on the surface of cancer cells and then permeate into cancer cells by endocytosis, and bind to proteins involved in cell death to inhibit cell growth. Such oligonucleotides have been reported to induce apoptosis due to cytostatic effects rather than cytotoxic effects (Paula J. Bates et al. The Journal of Biological Chemistry, 1999, 26369-26377; Bruna et al. FEBS journal, 2006, 1350-1361).

In addition, apart from the effect of inhibiting the growth of cancer cells, the oligonucleotides constituting G-quadruplexes are known to have various in vivo functions and regulatory functions, for example, U.S. Pat. No. 5,567,604 discloses that oligonucleotides constituting G-quadruplexes have antiviral activity, U.S. Pat. No. 6,994,959 discloses that oligonucleotides constituting G-quadruplexes have immunomodulatory activity, and US Patent Publication No. 2007-105805 discloses that oligonucleotides constituting G-quadruplexes have therapeutic effects on Huntington's disease (Cheryl A. Stoddart et al. Antimicrobial Agents and Chemotherapy, 1998, 2113-2115; Michael Skogen et al. BMC Neuroscience, 2006, 7:65).

Such oligonucleotides constituting G-quadruplexes induce apoptosis due to the cytostatic effect, so that the apoptosis rate is not relatively high. Therefore, it is difficult to administer the oligonucleotides in combination with a highly toxic chemotherapeutic agent (Paula J. Bates et al. Experimental and Molecular Pathology, 2009, 151-164; Christopher R. Ireson et al. Molecular Cancer Therapy, 2006, 2957-2962).

Korean Patent No. 10-0998365 discloses an example of improving an apoptotic effect by introducing a modified nucleic acid for treatment providing an apoptotic effect into oligonucleotides forming G-quadruplexes. However, the patent discloses neither oligonucleotide-modified nucleic acids according to the present invention nor their therapeutic effects on blood cancer and their apoptotic effects on drug-resistant blood cancer cells.

### PRIOR ART

### Patent Document

Korean Patent No. 10-0998365
U.S. Pat. No. 7,314,926
US Patent Laid-open No. 2007-105805
U.S. Pat. No. 5,567,604
U.S. Patent No. 6,994,959
US Patent Laid-open No. 2007-105805

### Non-Patent Document

(Non-patent Document 1) Christopher R. Ireson et al. Molecular Cancer Therapy, 2006, 2957-2962; Naijie Jing et al. Cancer Research, 2004, 6603-6609; Christophe Marchand et al. The Journal of Biological Chemistry, 2002, 8906-8911.
(Non-patent Document 2) Paula J. Bates et al. The Journal of Biological Chemistry, 1999, 26369-26377; Bruna et al. FEBS journal 2006 1350-1361]
(Non-patent Document 3) Cheryl A. Stoddart et al. Antimicrobial Agents and Chemotherapy, 1998, 2113-2115; Michael Skogen et al. BMC Neuroscience, 2006, 7:65.
(Non-patent Document 4) Paula J. Bates et al. Experimental and Molecular Pathology, 2009, 151-164; Christopher R. Ireson et al. Molecular Cancer Therapy, 2006, 2957-2962.

### DISCLOSURE

### Technical Problem

In view of the above problems, the present inventors introduced one or more of 1-β-D-arabinofuranosylcytosine, which is a modified nucleic acid having therapeutic efficacies to induce apoptosis through cytotoxic effect, into an oligonucleotide, which is rich in guanosine having a cytostatic effect and thus forms a G-quadruplex, in order to improve stability in blood and cell permeability, and to inhibit the growth of blood cancer cells more effectively, and to thereby induce cell death, and synthesized a novel oligonucleotide having such a structure and found, based on comparison in cytotoxicity, that the oligonucleotide has a dramatically improved effect of killing blood cancer cells, in particular, has excellent anti-cancer effect (level of nM) on drug-resistant blood cancer cells, which make treatment difficult because they do not respond to conventional therapeutic drugs for blood cancer and have excellent therapeutic efficacies on blood cancer. Finally, the present invention was completed based on this finding.

Therefore, it is one object of the present invention to provide a novel oligonucleotide-modified nucleic acid containing at least one 1-β-D-arabinofuranosylcytosine as a modified nucleic acid having a therapeutic effect and being rich in guanosine.

It is another object of the present invention to provide a composition for preventing, ameliorating or treating blood cancer, containing the oligonucleotide-modified nucleic acid or a pharmaceutically acceptable salt thereof as an active ingredient.

### Technical Solution

In one aspect, the present invention provides an oligonucleotide-modified nucleic acid containing a compound represented by the following Formula 2 to form a G-quadruplex structure:

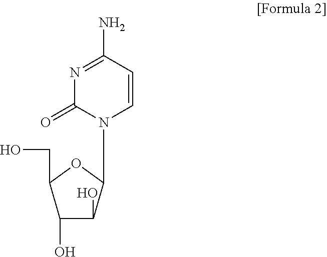

The oligonucleotide-modified nucleic acid may be represented by the following sequence:

Sequence 1)
GGTGGTGGTTNTGGTGGTGG;
Sequence 2)
GGTGGTGGTNNTGGTGGTGG;
Sequence 3)
NGGTGGTGGTTGTGGTGGTGG;
or
Sequence 4)
NNGGTGGTGGTTGTGGTGGTGG;

wherein G is guanosine or a guanosine derivative, T is thymine or a thymine derivative and N is 1-β-D-arabinofuranosylcytosine.

In another aspect, the present invention provides a composition for preventing, ameliorating or treating blood cancer containing the oligonucleotide-modified nucleic acid, or a pharmaceutically acceptable salt thereof as an active ingredient.

### Advantageous Effects

The novel oligonucleotide-modified nucleic acid according to the present invention is a compound with a novel structure capable of forming a G-quadruplex by incorporating at least one 1-β-D-arabinofuranosylcytosine and guanosine, and is thus useful as a preventive and therapeutic agent for blood cancer due to excellent apoptotic activity and anticancer therapeutic efficacies on blood cancer cells as well as drug-resistant blood cancer cells.

### DESCRIPTION OF DRAWINGS

The above and other objects, features and other advantages of the present invention will be more clearly understood from the following detailed description taken in conjunction with the accompanying drawings, in which:

FIG. 1 is a graph showing cytotoxic effects of an oligonucleotide-modified nucleic acid on AML cells (cytarabine-resistant MOLM13) with drug resistance to cytarabine (primary therapeutic agent for blood cancer);

FIG. 2 is a graph anti-proliferative effects of the oligonucleotide-modified nucleic acid on blood cancer cells (cytarabine-resistant MV-4-11) with drug resistance to cytarabine;

FIG. 3 shows a graph showing in vivo cytotoxicity of the oligonucleotide-modified nucleic acid in a blood cancer xenograft model (Luc-MOLM13);

FIG. 4 is an image showing in vivo cytotoxic efficacy of the oligonucleotide-modified nucleic acid in a blood cancer animal model (Luc-MOLM13);

FIG. 5 is a graph showing anti-proliferative efficacy and the survival rate of the oligonucleotide-modified nucleic acid on a blood cancer animal model (Luc-MOLM13);

FIG. 6 shows anticancer efficacy and the survival rate of the oligonucleotide-modified nucleic acid in a blood cancer syngeneic model (C1498);

FIG. 7 shows anticancer efficacy and the survival rate of the oligonucleotide-modified nucleic acid in a blood cancer syngeneic model (WEHI-3); and

FIG. 8 shows anti-proliferative efficacy of the oligonucleotide-modified nucleic acid on bone marrow mononuclear cells derived from relapsed/refractory patients.

### BEST MODE

Reference will now be made in detail to the preferred embodiments of the present invention, examples of which are illustrated in the accompanying drawings.

It should be understood that, in the specification, when the range is referred to regarding a parameter, the parameter encompasses all figures including end points disclosed within the range. For example, the range of “5 to 10” includes figures of 5, 6, 7, 8, 9, and 10, as well as arbitrary sub-ranges such as ranges of 6 to 10, 7 to 10, 6 to 9, and 7 to 9, and any figures, such as 5.5, 6.5, 7.5, 5.5 to 8.5 and 6.5 to 9, between appropriate integers that fall within the range. In addition, for example, the range of “10% to 30%” encompasses all integers that include figures such as 10%, 11%, 12% and 13%, as well as 30%, and any sub-ranges of 10% to 15%, 12% to 18%, or 20% to 30%, as well as any figures, such as 10.5%, 15.5% and 25.5%, between appropriate integers that fall within the range.

Hereinafter, the present invention will be described in detail.

In one aspect, the present invention is directed to an oligonucleotide-modified nucleic acid containing a compound represented by the following Formula 1 to form a G-quadruplex:

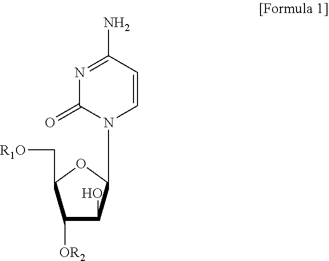

wherein R₁ is hydrogen or a phosphorus atom of the phosphate moiety of another nucleic acid, and R₂ is hydrogen or a phosphorus atom of the phosphate moiety of another nucleic acid.

In one aspect, the present invention is directed an oligonucleotide-modified nucleic acid wherein the compound is represented by the following Formula 2:

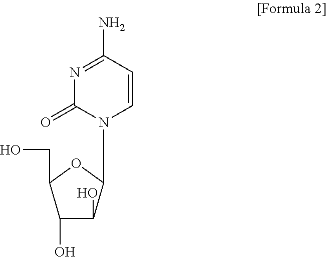

In one aspect of the present invention, the oligonucleotide-modified nucleic acid may be represented by the following sequence:

GaTbNc

wherein G is guanosine or a guanosine derivative, T is thymine or a thymine derivative, N is a modified nucleic acid of 1-β-D-arabinofuranosylcytosine or a derivative thereof, G, T and N are randomly arrayed by permutation, a is an integer selected from1 to 30, b is an integer selected from0 to 30, and c is an integer selected from1 to 30, with the proviso that a total of a, b and c does not exceed 60.

In one aspect of the present invention, the oligonucleotide-modified nucleic acid may be represented by the following sequence:

Sequence 1)
GGTGGTGGTTNTGGTGGTGG;
Sequence 2)
GGTGGTGGTNNTGGTGGTGG;
Sequence 3)
NGGTGGTGGTTGTGGTGGTGG;
or
Sequence 4)
NNGGTGGTGGTTGTGGTGGTGG;

In the Sequence above, G is guanosine or a guanosine derivative, T is thymine or a thymine derivative, and N is 1-β-D-arabinofuranosylcytosine as a modified nucleic acid.

In one aspect of the present invention, the guanosine or guanosine derivative may include one or more selected from 2-deoxy-guanosine, guanosine, 2′-O-methyl-guanosine, 2′-fluoro-guanosine, LNA (locked nucleic acid)-guanosine, D-deoxyguanosine and D-guanosine.

In another aspect, the present invention is directed to a composition for preventing, ameliorating or treating blood cancer containing at least one oligonucleotide-modified nucleic acid selected from those described above or a pharmaceutically acceptable salt thereof as an active ingredient.

In another aspect, the blood cancer includes at least one of non-Hodgkin's lymphoma, Hodgkin's lymphoma, multiple myeloma, leukemia, lymphoma, myelodysplastic syndrome (MDS), acute lymphoblastic leukemia, acute myelogenous leukemia, chronic lymphocytic leukemia, chronic myelogenous leukemia and solitary myeloma.

Hereinafter, embodiments of the present invention will be described in more detail.

In one aspect, the present invention provides a novel oligonucleotide-modified nucleic acid containing, as a modified nucleic acid having therapeutic efficacies, at least one 1-β-D-arabinofuranosylcytosine and being rich in guanosine.

In another aspect, the present invention provides a composition for preventing, ameliorating or treating blood cancer containing the oligonucleotide-modified nucleic acid or a pharmaceutically acceptable salt thereof as an active ingredient.

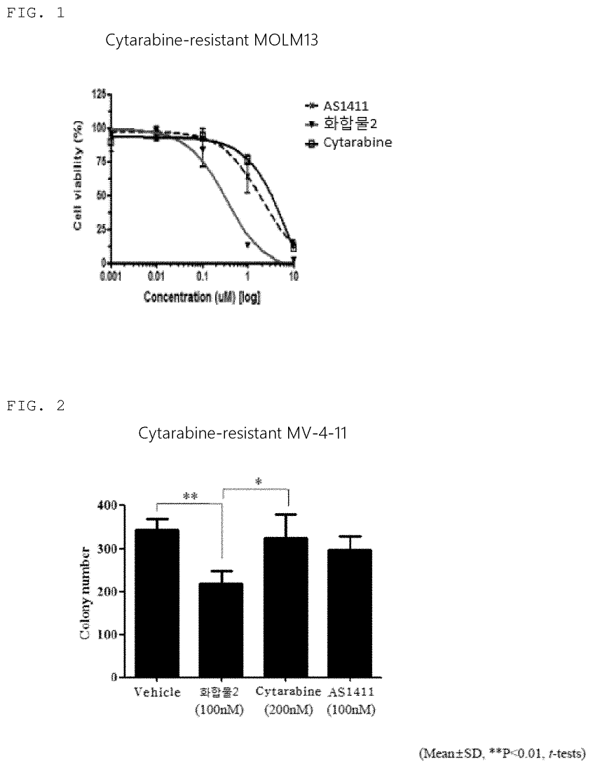

As used herein, the term “G-quadruplex” refers to an oligonucleotide that contains a great amount of one or more selected from 2-deoxy-guanosine, guanosine, 2′-O-methyl-guanosine, 2′-fluoro-guanosine, LNA (locked nucleic acid)-guanosine, D-deoxyguanosine, and D-guanosine representatively mentioned as guanosine (G) [see FIG. 1] and thus has a tetrahelical structure, in case of a certain sequence, based on Hoogsteen-type hydrogen bonds formed by four guanosines positioned in one plane. It is then synthesized such that a modified nucleic acid with a therapeutic effect is introduced into such a structure.

Oligonucleotides that form the G-quadruplex structure based on rich guanosine are known to bind more selectively to cancer cells and to inhibit growth of cancer cells through various mechanisms within cells.

The oligonucleotides forming G-quadruplexes, into which one or more modified nucleic acids are incorporated, are transferred to cancer cells. Thereafter, G-quadruplex exhibits an effect of inhibiting cell growth, and the modified nucleic acid, which exhibits a therapeutic effect when degraded by a nuclease, directly inhibits cell growth, thereby synergistically resulting in death of cancer cells. Since G-quadruplex-forming oligonucleotides only have an inhibitory activity against cell growth, when they are used alone, cell death rate is not relatively high and continuous treatment is needed over a certain period of time. However, it was identified by the present invention that incorporation of the modified nucleic acid having a therapeutic effect on blood cancer directly improves cell death effect, thereby rapidly increasing cell death rate.

In one aspect, the modified nucleic acid having therapeutic efficacies may be 1-β-D-arabinofuranosylcytosine.

In a specific embodiment, the cytidine-derived therapeutic modified nucleic acid (N) used herein is a cytidine derivative represented by the following Formula 2, wherein the nucleotide can be prepared in the form of phosphoramidite by an ordinary method [Oligonucleotides and Analogues: A Practical Approach 1991 Fritz Eckstein et al. IRL Press: Oxford], or can be produced to incorporate, in guanosine-rich oligonucleotide, nucleotide phosphoramidite purchased from Glen Research Corporation, Berry & Associates Inc., Okeanos Technologies, LLC., ChemGenes Corp., Proligo Corp., and the like, by solid phase synthesis using a DNA synthesizer in accordance with a conventionally reported method.

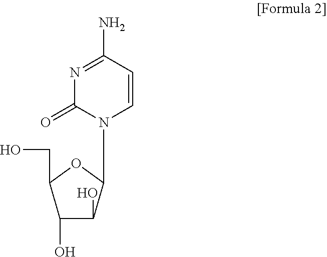

The oligonucleotide-modified nucleic acid can be produced by an ordinary method using the modified nucleic acid produced in the form of phosphoramidite with a solid phase synthesizer.

That is, the modified nucleic acid phosphoramidite is synthesized by dissolving in anhydrous acetonitrile and loading the same on a DNA synthesizer. The oligonucleotide-modified nucleic acid is synthesized using conventional synthesis and purification methods with reference to the guide (manual) provided by Glen Research Corporation and U.S. Pat. Nos. 5,457,187 and 5,614,505.

In addition, representative sequences of the oligonucleotide-modified nucleic acids that can be synthesized using the modified nucleic acid as described above are as follows:

Sequence 1)
GGTGGTGGTTNTGGTGGTGG
Sequence 2)
GGTGGTGGTNNTGGTGGTGG
Sequence 3)
NGGTGGTGGTTGTGGTGGTGG
or
Sequence 4)
NNGGTGGTGGTTGTGGTGGTGG

The compounds of the sequences, in which modified nucleic acids are incorporated at proper positions, exhibited excellent apoptotic effects on blood cancer cells and drug-resistant blood cancer cells, while exhibiting no significant effect on normal cells. It was also identified by animal experiments that the compounds of the sequences have excellent therapeutic efficacies against blood cancer.

Accordingly, the novel nucleotide modified nucleic acid can be completed by introducing the cytidine modified nucleic acid according to the present invention into the sequence of oligonucleotides having biological activity while forming G-quadruplexes through substitution.

The modified nucleic acid (N) having therapeutic efficacies is present as a compound represented by the following Formula 1 in the oligonucleotide-modified nucleic acid.

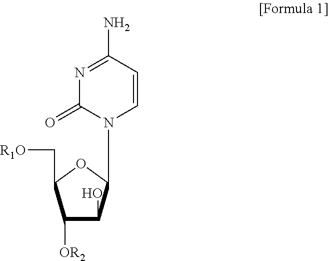

wherein R₁ is hydrogen or a phosphorus atom of the phosphate moiety of another nucleic acid and R₂ is hydrogen or a phosphorus atom of the phosphate moiety of another nucleic acid.

In the sequence, G represents guanosine and preferably includes one or more selected from 2-deoxy-guanosine, guanosine, 2′-O-methyl-guanosine, 2′-fluoro-guanosine, LNA (locked nucleic acid)-guanosine, D-deoxyguanosine, and D-guanosine, and N is 1-β-D-arabinofuranosylcytosine as cytidine-derived modified nucleic acid.

Conventional nucleoside-containing therapeutic agents having therapeutic effects may involve side effects such as systemic toxicity and drug resistance, and many cancer cells may have resistance to such therapeutic nucleoside anticancer drugs, which may require selection of other drugs. When the nucleoside having such therapeutic effects is incorporated in G-quadruplex oligonucleotides relatively more selective to cancer cells, the effects on the normal cells in vivo can be minimized and death (apoptosis) rates of even drug-resistant cancer cell tumors can be increased.

This G-quadruplex structure is more stable when potassium ions (K⁺) are present and thus has a stable structure for a long period of time under general physiological conditions such as in blood. Therefore, in the present invention, a G-quadruplex oligonucleotide containing a modified nucleic acid having a therapeutic effect is stabilized with a KCl solution with a certain concentration (30 to 70 mM) or the like before use.

The novel oligonucleotide-modified nucleic acid according to the present invention exhibits remarkably improved apoptotic effects in blood cancer cells and drug-resistant blood cancer cells, as compared with conventional therapeutic agents containing nucleosides having a therapeutic effect, and exhibits excellent anticancer therapeutic efficacies in evaluation of anti-cancer efficacy using animal models.

Accordingly, the present invention provides a composition preventing, ameliorating or treating cancer containing the oligonucleotide-modified nucleic acid or a pharmaceutically acceptable salt thereof as an active ingredient. The pharmacologically acceptable salts include, for example, metal salts, salts with organic bases, salts with inorganic acids, salts with organic acids, salts with basic or acidic amino acids, and the like. Suitable metal salts include: alkali metal salts such as sodium salts and potassium salts; alkaline earth metal salts such as calcium salts, magnesium salts and barium salts; and aluminum salts and the like. Suitable examples of salts with organic bases include salts of trimethylamine, triethylamine, pyridine, picoline, 2,6-lutidine, ethanolamine, diethanolamine, triethanolamine, cyclohexylamine, dicyclohexylamine, N,N-dibenzylethylenediamine, and the like. Suitable examples of salts with inorganic acids include salts of hydrochloric acid, hydrobromic acid, nitric acid, sulfuric acid, phosphoric acid and the like. Suitable examples of salts with organic acids include salts of formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid and the like.

Suitable examples of salts with basic amino acids include salts of arginine, lysine, ornithine and the like. Suitable examples of salts with acidic amino acid include salts of aspartic acid, glutamic acid and the like. Particularly preferred salts include, when the compound has an acidic functional group therein, inorganic salts such as alkali metal salts (for example, sodium salts and potassium salts) and alkaline earth metal salts (for example, calcium salts, magnesium salts and barium salts), and organic salts such as ammonium salts. When the compound has a basic functional group therein, particularly preferred salts include salts with inorganic acids such as hydrochloric acid, hydrobromic acid, nitric acid, sulfuric acid and phosphoric acid, and salts with organic acids such as acetic acid, phthalic acid, fumaric acid, oxalic acid, tartaric acid, maleic acid, citric acid, succinic acid, methanesulfonic acid and p-toluenesulfonic acid.

The composition for preventing, ameliorating or treating cancer containing the oligonucleotide-modified nucleic acid according to the present invention may contain a pharmaceutically acceptable carrier in addition to the active ingredient. In case of an injection solution, the pharmaceutically acceptable carrier may be a mixture of a preservative, an isotonic agent, an anesthetic agent, a solubilizing agent, a buffer, a stabilizing agent or the like. For oral administration, the pharmaceutically acceptable carrier may be a solubilizing agent, a binder, an excipient, a dispersant, a stabilizing agent, a suspending agent, a color disintegrating agent, a lubricant, a flavor or the like. For topical administration, the pharmaceutically acceptable carrier may be a base, an excipient, a lubricant, a preservative or the like.

The composition formulation according to the present invention may be variously prepared as a mixture with the pharmaceutically acceptable carrier. In addition, the composition may be prepared in the form of a unit dosage ampoule or a plurality of dosage forms for injection and in the form of a tablet, elixir, capsule, suspension, troche, wafer, syrup or the like for oral administration, and may be formulated into a tablet, a pill, a capsule, a preparation with sustained-release or the like.

Meanwhile, examples of suitable carriers, excipients and diluents for formulation include microcrystalline cellulose, xylitol, erythritol, methyl cellulose, polyvinyl pyrrolidone, starch, acacia, alginate, gelatin, lactose, dextrose, sucrose, propyl hydroxybenzoate, cellulose, water, methyl hydroxybenzoate, magnesium stearate, talc, sorbitol, mannitol, maltitol, calcium phosphate, calcium silicate, mineral oil or the like.

The term “administration” used herein means introduction of a certain substance to a patient by any suitable method. The administration route of the active ingredient may be any general route enabling a drug to be delivered to a target tissue. Examples of the administration route include, but are not limited to, intravenous, subcutaneous, oral, intramuscular, intraperitoneal, intrapulmonary, rectal, local, intranasal and intradermal administrations. However, because oligonucleotides are digested when administered via oral administration, the composition for oral administration must be prepared so that it can be decomposed and absorbed in gastrointestinal tract. Preferably, it may be administered by injection or via an internasal route.

The content of the active ingredient in the preparation according to the present invention can be appropriately selected depending on absorptivity of the active ingredient in the body, inactivation ratio, excretion rate, age, gender and conditions of the user and the like. The dose of the active ingredient of the present invention may be 1 to 1,000 mg/kg, preferably 3 to 100 mg/kg, and can be continuously administered 1 to 3 times a day or via infusion for a predetermined period of time.

Hereinafter, the present invention will be described in detail with reference to the following Examples and is not limited thereto.

### EXAMPLE 1

### Synthesis of Novel Oligonucleotide-Modified Nucleic Acid

DNA synthesis was performed on a 1 μmol scale using a conventional solid phase DNA synthesizer. Regarding the phosphoramidites used for synthesis, deoxyguanosine, thymidine, and 1-β-D-arabinofuranosylcytosine phosphoramidite were purchased from Glen Research Corporation, which were dissolved at a concentration of 0.067 M in dry acetonitrile, and loaded on a solid phase DNA synthesizer from Polygene Inc. The reaction proceeds in the direction of 3′→5′, and the 3′ hydroxyl group of the first nucleotide is attached to the resin. During addition of one base, four-step chemical reactions including 5′-terminus detritylation, coupling of a new base, capping of a uncoupled DNA chain, and oxidation of the phosphate group were repeated. After the reaction was completed, the protecting group was removed, and the synthesized CPG resin was immersed in aqueous ammonia and allowed to stand at 55° C. for 5 hours. Then, the aqueous ammonia was dried to obtain a white powder. Purification was performed by increasing a 1M NaCl solution to 5 to 70% in an HPLC system using a Waters anion exchange HPLC column. The main peaks were collected, 100% ethanol was added thereto, and the oligonucleotides were precipitated and then dried. As a result, oligonucleotides having a purity of 85% or more were obtained by HPLC, and molecular weights were measured using ESI-LC-MS (Q-TRAP 2000 ESI-MS) to determine whether or not synthesis was successful.

Oligonucleotide-modified nucleic acids can be produced in the same manner as above, with respect to oligonucleotide sequences which were already reported for the purpose of therapeutic treatment or a variety of sequences that exhibit physiological activity, while forming G-quadruplexes, in addition to the sequences shown in the following Table 1.

TABLE 1

| No. of | | | | | |
|---|---|---|---|---|---|
| Compound | N | G | T | Sequence | LC/MS |
| Compound | 1-β-D- | 2′-deoxy- | Thymidine | GGTGGTGGTTNTGGTGGTGG | 6323.1 |
| 1 | arabinofuranosylcytosine | guanosine | | | |
| Compound | 1-β-D- | 2′-deoxy- | Thymidine | GGTGGTGGTNNTGGTGGTGG | 6324.1 |
| 2 | arabinofuranosylcytosine | guanosine | | | |
| Compound | 1-β-D- | 2′-deoxy- | Thymidine | NGGTGGTGGTTGTGGTGGTGG | 6652.3 |
| 3 | arabinofuranosylcytosine | guanosine | | | |
| Compound | 1-β-D- | 2′-deoxy- | Thymidine | NNGGTGGTGGTTGTGGTGGTGG | 6957.5 |
| 4 | arabinofuranosylcytosine | guanosine | | | |

### EXAMPLE 2

### Production of Novel Oligonucleotide-Modified Nucleic Acid Solution

Each oligonucleotide-modified nucleic acid was diluted in a 10 mM Tris-HCl (pH 7.4) solution to a final concentration of 100 μM. The diluted solution was allowed to stand at 94° C. for 5 minutes and then the tube was allowed to stand on ice. The resulting solution was added with a 2 M KCl solution such that a final concentration of the KCl solution became 50 mM, allowed to stand at 60° C. for 3 hours and was slowly cooled to room temperature. Each oligonucleotide was diluted in a 10 mM Tris-HCl (pH 7.4) solution to a concentration of 10 μM, and was allowed to stand at 94° C. for 5 minutes, and the tube was allowed to stand on ice. The resulting solution was added with a 2 M KCl solution such that the final concentration of the KCl solution became 50 mM, and allowed to stand at 60° C. for 3 hours and then slowly cooled to room temperature, which was then used for the present Example.

### EXPERIMENTAL EXAMPLE 1

### Cytotoxic Efficacy in Blood Cancer Cells and Drug-Resistant Blood Cancer Cells In Vitro

On the day before the experiment, 190 μl of a culture medium (at a concentration of 10⁴ to 10⁵ cells/ml) containing blood cancer cells such as HL60, MV-4-11, CCRF-CEM, MOLT-4 and MOLM-13 cell lines, and drug-resistant blood cancer cells such as azacitidine (AZA-1)-resistant MOLM13, decitabine (Dec-5)-resistant MOLM13 and cytarabine (AraC-1)-resistant MOLM13 cell lines, and normal cells such as WI38, CCD-18co and Fa2N4 cells were seeded on a 96-well plate. The next day, 10 pl of the oligonucleotide-modified nucleic acid solution prepared in Example 2 was added and then cultured for 5 days.

Five days after drug treatment, MTT assay was performed to measure cytotoxicity of the cell lines [see JBC 1999, 26369].

Results of measurement of cytotoxicity by novel oligonucleotide-modified nucleic acids according to the present invention showed, as can be seen from Table 2, IC₅₀ of the novel oligonucleotide-modified nucleic acids, i.e., Compounds 1, 2, 3, and 4 of the present invention, exhibited superior cytotoxic effects on various blood cancer cell lines [Table 2].

In addition, it could be seen that the novel oligonucleotide-modified nucleic acids according to the present invention exhibited superior cytotoxic effects on various drug-resistant blood cancer cells as well [Table 3].

In addition, it could be seen that the novel oligonucleotide-modified nucleic acids according to the present invention exhibited superior cytotoxic effects on various blood cancer cells as well as various drug-resistant blood cancer cells, while they had almost no effect on normal cells [Table 4].

The results of measurement of cytotoxic effects are shown in Tables 2 to 4 below. In addition, cytotoxic effects of the oligonucleotide-modified nucleic acids according to the present invention on drug-resistant blood cancer cells are identified and shown in FIGS. 1 and 2.

TABLE 2
Cytotoxic effects on blood cancer cells

IC₅₀ (μM)

| | HL60 | CCRF-CEM | MOLT-4 | MOLM13 |
|---|---|---|---|---|
| Compound 1 | 0.03 | 0.01 | 0.021 | 0.09 |
| Compound 2 | 0.011 | 0.006 | 0.02 | 0.089 |
| Compound 3 | 1.15 | 0.08 | 0.036 | 0.195 |
| Compound 4 | 1.45 | 0.038 | 0.035 | 0.15 |

TABLE 3
Cytotoxic effects on drug-resistant blood cancer cells

| | IC₅₀ (μM) | | | | |
|---|---|---|---|---|---|
| | Cytarabine- | Cytarabine- | Cytarabine- | Azacitidine- | Decitabine- |
| | resistant | resistant | resistant | resistant | resistant |
| | MOLM13 | HL-60 | MV-4-11 | MOLM13 | MOLM13 |
| Compound 2 | 0.147 | 1.625 | 3.3 | 0.1347 | 0.1016 |
| Cytarabine | >10 | >30 | >20 | 3.5000 | 3.4930 |

TABLE 4
Cytotoxic effects on normal cells

| | IC₅₀ (μM) | | |
|---|---|---|---|
| | WI38 | CCD-18co | Fa2N4 |
| Compound 2 | >10 | >5 | >10 |

### EXPERIMENTAL EXAMPLE 2

### Anticancer Efficacy In Human-Derived Blood Cancer Cell (Luc-MOLM13) Xenograft Mouse Model In Vivo

In order to identify efficacies on human-derived blood cancer cells in animals, blood cancer was induced with NOD-SCID mice to evaluate anticancer efficacies in vivo.

First, the induction of blood cancer was carried out specifically as follows. The experimental group consisted of a vehicle group (8 mice) and a drug administration group (9 mice).

Luc-MOML-13 cells (human MDS/AML cell lines) were intravenously injected at a density of 10⁷ (cells/mouse) into 8-week-old NOD-SCID mice. As the administered drug, Compound 2 was dissolved in potassium buffer at a concentration of 2 mM, and the solution was allowed to stand at 94° C. for 5 minutes and slowly cooled down to room temperature for 3 hours, which was then used for the present Example.

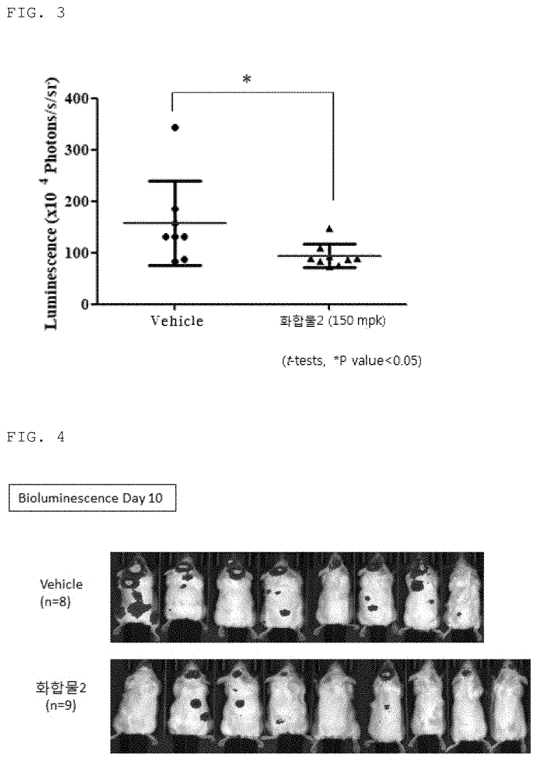

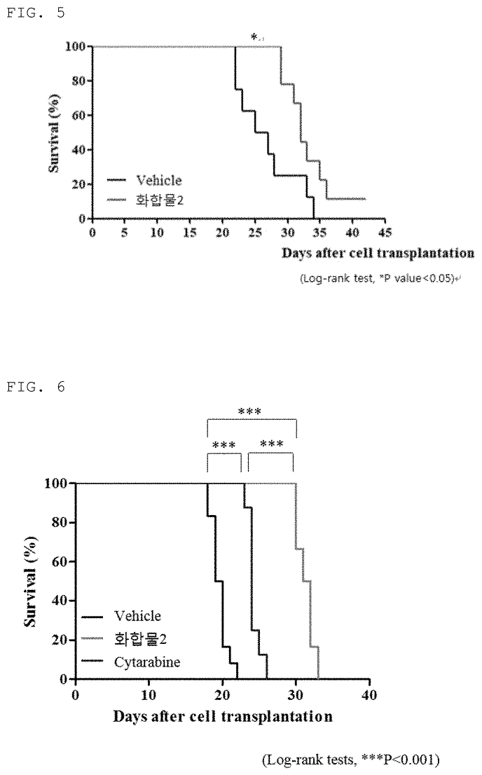

The drug was administered intravenously (150 mg/kg) daily for two weeks, five times a week from the second day after cell inoculation, and the survival rate was monitored. The results are shown in FIG. 5. In addition, after 10 days since the start of drug administration, the cytotoxic effect of blood cancer cells was compared with that of the vehicle group by biomedical imaging and the results are shown in FIGS. 3 and 4.

As can be seen from FIGS. 3 and 4, the blood cancer cells of the drug administration group were significantly reduced as compared to the vehicle group.

As can be seen from FIG. 5, the survival rate of the drug administration group was significantly improved as compared to the vehicle group.

### EXPERIMENTAL EXAMPLE 3

### Anticancer Efficacy in AML (C1498) Syngeneic Mouse Model In Vivo

In order to identify in vivo efficacies on blood cancer cells with an immune system, the survival rate of mice was monitored after transplanting mouse-derived blood cancer cells (C1498) into C57BL/6 mice.

The induction of blood cancer was carried out specifically as follows. The experimental group consisted of a vehicle group (8 mice), a drug administration group (9 mice) and a control group (8 mice). Compound 2 was injected as the drug administration group at 300 mg/kg, and cytarabine was injected as the positive control group at 30 mg/kg (cytarabine in the same amount as 300 mg/kg of Compound 2).

C1498 cells (mouse AML cell line) were intravenously injected at a density of 5×10⁵ (cells/mouse) into 8-week-old C57BL/6 mice. As the administered drug, Compound 2 was dissolved in potassium buffer at a concentration of 2 mM, and the solution was allowed to stand at 4° C. for 6 hours.

The drug was subcutaneously injected twice daily for two weeks from the second day after cell inoculation, and survival rate was monitored. The results are shown in FIG. 6.

As can be seen in FIG. 6, the survival rate of the drug administration group was significantly improved as compared to the vehicle group and the survival rate of the drug administration group was far superior to that of the positive control group.

### EXPERIMENTAL EXAMPLE 4

### Anticancer Efficacy in AML (WEHI-3) Syngeneic Mouse Model In Vivo

In order to identify in vivo efficacies on AML cells with an immune system, the viability of the mice was evaluated by transplanting mouse-derived blood cancer cells (WEHI-3) into Balb/c mice.

The induction of blood cancer was carried out specifically as follows. The experimental group consisted of a vehicle group (7 mice), a drug administration group (7 mice) and a control group (7 mice). Compound 2 was injected as the drug administration group at 300 mg/kg, and cytarabine was injected as the positive control group at 30 mg/kg (cytarabine in the same amount as 300 mg/kg of Compound 2).

WEHI-3 cells (mouse AML cell line) were intravenously injected at a density of 5×10⁵ (cells/mouse) into 8-week-old Balb/c mice. As the administered drug, Compound 2 was dissolved in potassium buffer at a concentration of 2 mM, and the solution was allowed to stand at 4° C. for 6 hours.

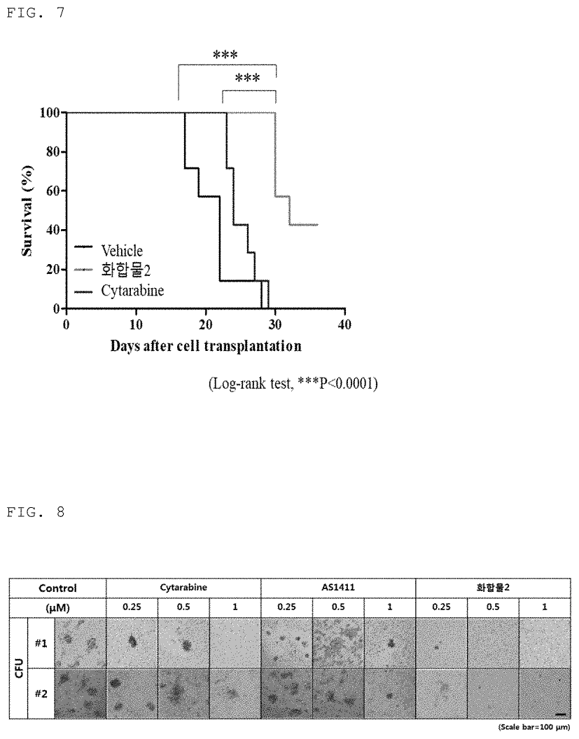

The drug was subcutaneously injected twice daily for two weeks from the second day after cell inoculation, and viability was measured. The results are shown in FIG. 7.

As can be seen in FIG. 7, the survival rate of the drug administration group was significantly improved as compared to the vehicle group and the survival rate of the drug administration group was far superior to that of the positive control group.

### EXPERIMENTAL EXAMPLE 5

### Anticancer Efficacies in Bone Marrow Mononuclear Cells Derived from Relapsed/refractory Patients

In order to identify the anticancer efficacy in actual patient blood cancer cells, bone marrow mononuclear cells derived from relapsed/refractory (R/R) acute myeloid leukemia patients were used for colony formation with respect to Compound 2 and cytarabine (AS1411), respectively. The cells were provided after IRB approval at the Asan Medical Center in Seoul. Bone marrow mononuclear cells were seeded onto colony forming media and then treated with 0.25 μM, 0.5 μM, and 1 μM of each drug, and cultured for 16 days, and the number of colonies was counted.

As can be seen from FIG. 8, the positive control drug with the same concentration had an insufficient therapeutic effect on in the relapsed/refractory blood cancer cells, whereas Compound 2 had a significantly excellent anticancer effect.

### EXPERIMENTAL EXAMPLE 6

### Toxicity Tests

Toxicity tests on the active ingredients of the present invention were carried out as follows.

Compound 2 was dissolved in potassium buffer, and the solution was injected at 1 g/kg into mice (10 mice per group) and then observed for 7 days. As a result, no mice died, which means that the lethal dose (LD₅₀) was at least 1 g/kg.

In addition, when the drug was administered to normal mice with no blood cancer under the same conditions as in Experimental Example 4, the normal leukocyte was rapidly decreased in the control group, while the drug administration group to which Compound 2 was administered maintained relatively high white blood cell counts (vehicle group mean: 6.29, vehicle group range: 4.70-8.97, drug administration group mean: 4.07, drug administration group range: 3.36-4.77, control group mean: 2.34, control group range: 0.95-4.31), thereby exhibiting excellent safety. In summary, the results of Experimental Examples 3 to 5 showed that Compound 2 specifically kills blood cancer cells and minimizes the death of normal blood cells, thus exhibiting excellent characteristics as a therapeutic agent targeting blood cancer.

### PREPARATION EXAMPLE 1

### Preparation of Injection Solution

An injection solution containing 10 mg of Compound 2 was prepared by the following method.

1 g of Compound 2, 0.6 g of sodium chloride and 0.1 g of ascorbic acid were dissolved in distilled water to prepare a 100 ml solution. A bottle was filled with the solution and sterilized by heating at 20° C. for 30 minutes.

The ingredients for the injection solution are as follows:

1 g of active ingredient

0.6 g of sodium chloride

0.1 g of ascorbic acid

Appropriate amount of distilled water

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An oligonucleotide-modified nucleic acid comprising a compound represented by the following Formula 1 to form a G-quadruplex structure: 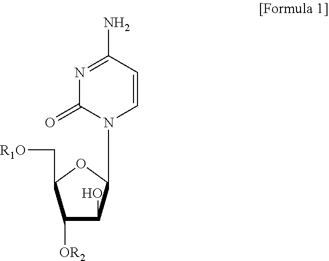
wherein R1 is hydrogen or a phosphorus atom of the phosphate moiety of another nucleic acid; and
R2 is hydrogen or a phosphorus atom of the phosphate moiety of another nucleic acid.

2. The oligonucleotide-modified nucleic acid according to claim 1, wherein the compound is represented by the following Formula 2;

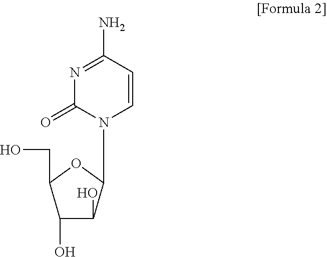

3. The oligonucleotide-modified nucleic acid according to claim 2, wherein the oligonucleotide-modified nucleic acid is represented by the following sequence:
GaTbNc
wherein G is guanosine or a guanosine derivative;
T is thymine or a thymine derivative;
N is 1-β-D-arabinofuranosylcytosine as a modified nucleic acid;
G, T and N are randomly arrayed by permutation; and
a is an integer selected from 1 to 30, b is an integer selected from 0 to 30, and c is an integer selected from 1 to 30, with the proviso that a total of a, b and c does not exceed 60.

4. The oligonucleotide-modified nucleic acid according to claim 3, wherein the oligonucleotide-modified nucleic acid is represented by the following sequence:
Sequence 1)
GGTGGTGGTTNTGGTGGTGG;
Sequence 2)
GGTGGTGGTNNTGGTGGTGG;
Sequence 3)
NGGTGGTGGTTGTGGTGGTGG;
or
Sequence 4)
NNGGTGGTGGTTGTGGTGGTGG;
wherein G is guanosine or a guanosine derivative;
T is thymine or a thymine derivative; and
N is 1-β-D-arabinofuranosylcytosine as a modified nucleic acid.

5. The oligonucleotide-modified nucleic acid according to claim 3, wherein the guanosine or guanosine derivative comprises one or more selected from 2-deoxy-guanosine, guanosine, 2′-O-methyl-guanosine, 2′-fluoro-guanosine, LNA (locked nucleic acid)-guanosine, D-deoxyguanosine and D-guanosine.

6. A composition for preventing, ameliorating or treating blood cancer comprising the oligonucleotide-modified nucleic acid according claim 1, or a pharmaceutically acceptable salt thereof as an active ingredient.

7. The composition for preventing, ameliorating or treating blood cancer according to claim 6, wherein the blood cancer comprises at least one of non-Hodgkin's lymphoma, Hodgkin's lymphoma, multiple myeloma, leukemia, lymphoma, myelodysplastic syndrome (MDS), acute lymphoblastic leukemia, acute myelogenous leukemia, chronic lymphocytic leukemia, chronic myelogenous leukemia and solitary myeloma.

8. A composition for preventing, ameliorating or treating blood cancer resistant to at least one of cytarabine, decitabine and azacitidine, as DNA synthetase or DNA methyltransferase inhibitors, the composition comprising the oligonucleotide-modified nucleic acid according to claim 1, or a pharmaceutically acceptable salt thereof as an active ingredient.

9. A composition for preventing, ameliorating or treating acute myelogenous leukemia having high apoptosis induction effects in bone marrow mononuclear cells derived from recurrent/intractable acute myelogenous leukemia patients, the composition comprising the oligonucleotide-modified nucleic acid according to claim 1, or a pharmaceutically acceptable salt thereof as an active ingredient.

10. A composition for ameliorating or treating blood cancer having higher apoptosis induction effects in cancer cells than in normal cells, the composition containing the oligonucleotide-modified nucleic acid according to claim 1, or a pharmaceutically acceptable salt thereof as an active ingredient.
